(19) United States
(12) Patent Application Publication
(10) Pub. No.: US 2023/0152330 A1
(43) Pub. Date: May 18, 2023

(54) DETECTION AND TREATMENT OF IDIOPATHIC PULMONARY FIBROSIS

(71) Applicant: AmMax Bio, Inc., Redwood City, CA (US)

(72) Inventors: Kirk William Johnson, Moraga, CA (US); Michael Huang, Redwood City, CA (US)

(21) Appl. No.: 18/056,033

(22) Filed: Nov. 16, 2022

Related U.S. Application Data
(60) Provisional application No. 63/280,526, filed on Nov. 17, 2021.

(51) Int. Cl. G01N 33/68

(52) U.S. Cl. CPC G01N 33/6872; G01N 2800/12; G01N 2800/52; G01N 2333/7153

(57) ABSTRACT

The present disclosure relates generally to compositions and methods for diagnosing and treating idiopathic pulmonary fibrosis (IPF). Also, methods for identifying IPF disease severity and likelihood for disease progression. An IPF patient can be identified when the CSF1 concentration or soluble CSF1R concentration in a blood sample is decreased as compared to a reference blood sample from a reference human subject not having IPF, and/or when the soluble CSF1R concentration in a bronchoalveolar lavage (BAL) fluid sample is increased as compared to a reference BAL fluid sample from the reference human subject. Once identified, the patient can be treated with, for instance, CSF1 or CSF1R inhibitors, such as antibodies. Moreover, the IPF patient soluble CSF1R level in blood or BAL samples can also be used to assess the severity of disease, and to both monitor disease progression and treatment outcomes.

### CROSS REFERENCE TO RELATED APPLICATIONS

This application claims the benefit under 35 U.S.C. § 119(e) of the U.S. Provisional Application Ser. No. 63/280,526, filed Nov. 17, 2021, the content of which is hereby incorporated by reference in its entirety.

### BACKGROUND

Idiopathic pulmonary fibrosis (IPF) is a serious chronic disease that affects the tissue surrounding the air sacs, or alveoli, in the lung. This condition occurs when that lung tissue becomes thick and stiff for unknown reasons. Over time, these changes can cause permanent scarring in the lungs, called fibrosis, that make it progressively more difficult to breathe.

The risk for IPF is higher for smokers and those having a family history of IPF. Additional risk factors include acid reflux disease (GERD) and certain viral infections. The underlying mechanism involves scarring of the lungs. The most common symptoms of IPF are shortness of breath and cough. Some people may not have symptoms at first, but signs and symptoms can develop and get worse as the disease progresses.

The way IPF advances varies from person to person, and scarring may happen slowly or quickly. In some people, the disease stays the same for years. In other people, the condition rapidly declines. Many people with IPF also experience acute exacerbations, where symptoms suddenly become much more severe. Other complications of IPF include pulmonary hypertension and respiratory failure, which happens when the lungs cannot deliver enough oxygen into the bloodstream without support. This prevents the brain and other organs from getting the oxygen they need.

About 5 million people are affected globally. The disease newly occurs in about 12 per 100,000 people per year. Those in their 60s and 70s are most commonly affected. Males are affected more often than females. Average life expectancy following diagnosis is about four years.

There is currently no cure for IPF. Certain medicines may slow the progression of IPF, which may extend the lifespan and improve the quality of life for people who have the disease.

An earlier diagnosis of IPF is a prerequisite for earlier treatment and, potentially, improvement of the long-term clinical outcome of this progressive and ultimately fatal disease. Diagnosis of IPF is not straight forward and requires ruling out other potential causes. It may be supported by a HRCT scan or lung biopsy which show usual interstitial pneumonia (UIP).

### SUMMARY

The instant inventors made the discovery that certain proteins have higher or lower concentrations in blood or bronchoalveolar lavage (BAL) fluid samples from idiopathic pulmonary fibrosis (IPF) patients as compared to healthy subjects. Moreover, elevated levels of some (e.g. soluble CSF1R) correlate with disease severity and disease progression. These proteins, including CSF1 and soluble CSF1R, therefore, can be used for diagnosis, prognosis, patient selection and treatment monitoring.

One embodiment of the present disclosure provides a method for identifying a patient as likely having idiopathic pulmonary fibrosis (IPF), comprising measuring the concentration of CSF1 or soluble CSF1R in a blood (e.g. plasma) or a bronchoalveolar lavage (BAL) fluid sample from a patient; and identifying the patient as likely having IPF when the CSF1 concentration or soluble CSF1R concentration in the blood sample is decreased as compared to a reference blood sample from a reference human subject not having IPF, or when the soluble CSF1R concentration in the BAL fluid sample is increased as compared to a reference BAL fluid sample from a reference human subject not having IPF.

In some embodiments, the identified patient is treated, with a therapy such as an oxygen therapy, pulmonary rehabilitation, or an agent selected from the group consisting of interferon gamma-1β, bosentan, ambrisentan, an anticoagulant, pirfenidone, N-Acetylcysteine (NAC), nintedanib, a CSF1 inhibitor, and a CSF1R inhibitor.

In some embodiments, the patient is identified as likely having IPF when the soluble CSF1R concentration in the BAL fluid sample is greater than 1500 pg/mL. In some embodiments, the patient is identified as likely having IPF when the soluble CSF1R concentration in the BAL fluid sample is greater than 1800 pg/mL, preferably greater than 2000 pg/mL.

In some embodiments, the patient is identified as likely having IPF when the soluble CSF1R concentration in the blood sample is lower than 200 ng/mL. In some embodiments, the patient is identified as likely having IPF when the soluble CSF1R concentration in the blood sample is lower than 180 ng/mL, preferably lower than 160 ng/mL.

In some embodiments, the patient is identified as likely having IPF when the CSF1 concentration in the blood sample is lower than 1.8 pg/mL. In some embodiments, the patient is identified as likely having IPF when the CSF1 concentration in the blood sample is lower than 1.6 pg/mL, preferably lower than 1.5 pg/mL.

Another embodiment provides a method for treating an idiopathic pulmonary fibrosis (IPF), comprising administering a CSF1 inhibitor or a CSF1R inhibitor to a patient that (a) has a blood CSF1 concentration lower than a reference blood CSF1 concentration from a reference human subject not having IPF, (b) has a blood soluble CSF1R concentration lower than a reference blood soluble CSF1R concentration from a reference human subject not having IPF, or (c) has a BAL fluid soluble CSF1R concentration higher than a reference BAL fluid soluble CSF1R concentration from a reference human subject not having IPF.

Yet another embodiment provides a method for monitoring the disease progression in an idiopathic pulmonary fibrosis (IPF) patient, comprising measuring the concentration of soluble CSF1R in a blood (e.g. plasma) or a bronchoalveolar lavage (BAL) fluid sample from the IPF patient, and determining that the IPF has worsened when the concentration of soluble CSF1R in the blood sample has increased, or when the concentration of the soluble CSF1R in the BAL fluid sample has increased, as compared to an earlier measurement for the patient.

Yet another embodiment provides a method for monitoring the effect of a treatment of an idiopathic pulmonary fibrosis (IPF) patient, comprising measuring the concentration of CSF1 or soluble CSF1R in a blood or a bronchoalveolar lavage (BAL) fluid sample from the IPF patient, and determining that the treatment is effective when the concentration of CSF1 or soluble CSF1R in the blood sample has increased, or when the concentration of the soluble CSF1R in the BAL fluid sample has decreased, as compared to an earlier measurement for the patient during or before the treatment.

Another embodiment provides a method for characterizing the severity of idiopathic pulmonary fibrosis (IPF) in an IPF patient, comprising measuring the soluble CSF1R level in a plasma sample from the patient, and characterizing the severity of the IPF including likelihood to progress towards death or >10% FVC loss or lung transplant when the CSF1R level is higher than a reference level. In some embodiments, the threshold level is 100 pg/mL (i.e., disease progression is worse for IPF patients with login-transformed plasma sol.CSF1R pg/ml levels >2.0 vs those </=2.0). The BAL fluid soluble CSF1R levels can likewise be used. In some embodiments, the reference level for the BAL fluid level is 2000 pg/mL (i.e. survival is worse in IPF patients with BAL sol.CSF1R levels greater than ˜2000 pg/ml).

### BRIEF DESCRIPTION OF THE DRAWINGS

FIG. 1 shows the levels of IL-34 and CSF1 in plasma samples from IPF patients in comparison to healthy subjects.

FIG. 2 shows the levels of IL-34 and CSF1 in BAL fluid samples from IPF patients in comparison to healthy subjects.

FIG. 3 shows the levels of soluble CSF1R in plasma and BAL fluid samples from IPF patients in comparison to healthy subjects.

FIG. 4 shows that the IPF patients having high or low soluble CSF1R levels had different survival rates.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

Colony stimulating factor 1 (CSF-1), also known as macrophage colony stimulating factor (M-CSF), is a cytokine produced by a variety of cells, including macrophages, endothelial cells and fibroblasts. CSF-1 is composed of two “monomer” polypeptides, which form a biologically active dimeric CSF-1 protein. CSF-1 exists in at least three mature forms due to alternative RNA splicing (see, Cerretti et al. Molecular Immunology, 25:761 (1988)). The three forms of CSF-1 are translated from precursors, which encode polypeptide monomers of 256 to 554 amino acids, having a 32 amino acid signal sequence at the amino terminal and a putative transmembrane region of approximately 23 amino acids near the carboxyl terminal. The precursor peptides are subsequently processed by amino terminal and carboxyl terminal proteolytic cleavages to release mature CSF-1. Residues 1-149 of all three mature forms of CSF-1 are identical and are believed to contain sequences essential for biological activity of CSF-1. CSF-1 monomers are dimerized in vivo via disulfide-linkage and are glycosylated. CSF-1 belongs to a group of biological agonists that promote the production of blood cells. Specifically, it acts as a growth and differentiation factor for bone marrow progenitor cells of the mononuclear phagocyte lineage.

Colony stimulating factor 1 receptor (referred to herein as CSF1R; also referred to as FMS, FIM2, C-FMS, or CD115) is a single-pass transmembrane receptor with an N-terminal extracellular domain (ECD) and a C-terminal intracellular domain with tyrosine kinase activity. CSF1R belongs to the type III protein tyrosine kinase receptor family, and binding of CSF1 or the interleukin 34 ligand induces homodimerization of the receptor and subsequent activation of receptor signaling. CSF1R-mediated signaling is crucial for the differentiation and survival of the mononuclear phagocyte system and macrophages in particular.

Soluble CSF1R (sol. CSF1R or sCSF1R) refers to be the extracellular domain of CSF1R that can be cleaved off from the full-length CSF1R protein. The precursor human CSF1R (e.g., NP_001275634.1) is 972 amino acid residues long, including a 19-amino acid signal peptide (1-19), an extracellular domain that includes five immunoglobulin (Ig) domains (20-517), a transmembrane domain (518-538), and an intracellular domain (539-972) that includes the Catalytic domain of the Protein Tyrosine Kinase (PTKc). A cleavage between one or more Ig domains of the extracellular domain and a more C-terminal domain (e.g., the transmembrane domain or PTKc) produces the soluble CSF1R.

Interleukin 34 (IL-34) is a cytokine that increases growth or survival of monocytes and elicits its activity by binding the CSF1R. IL-34 is a tissue-restricted ligand of CSF1R required for the development of Langerhans cells and microglia.

The disclosure further provides diagnostic, prognostic and therapeutic methods, which are based, at least in part, on determination of the expression level of a gene of interest identified herein.

For example, information obtained using the diagnostic assays described herein is useful for determining if a subject is likely suffering from a disease (e.g., IPF) or likely to develop the disease, or is suitable for a treatment. Based on the diagnostics/prognostic information, a doctor can recommend a therapeutic protocol.

It is to be understood that information obtained using the diagnostic assays described herein may be used alone or in combination with other information, such as, but not limited to, behavior assessment, genotypes or expression levels of other genes, clinical chemical parameters, histopathological parameters, or age, gender and weight of the subject.

### Diagnostic and Prognosis Methods

There is currently no cure for idiopathic pulmonary fibrosis (IPF). Early diagnosis and disease prevention, therefore, are important for disease management. Diagnosis of IPF, however, is challenging, as it requires ruling out other potential causes.

Attempts have been made to identify suitable biomarkers for IPF diagnosis. For instance, Fraser et al. 2021 (Front Immunol, 2021 Mar. 5; 12:623430) has recently reported elevated serum CSF1 levels in IPF patients (N=37, ˜1000 pg/ml) vs controls (N=28, ˜250 pg/ml). Likewise, Baran 2007 (Am J Respir Crit Care Med., 2007 Jul. 1; 176(1):78-89) reported that the CSF1 levels in bronchoalveolar lavage (BAL) fluids were increased in IPF patients (N˜25, 155 pg/ml) as compared to controls (N˜25, 49 pg/ml).

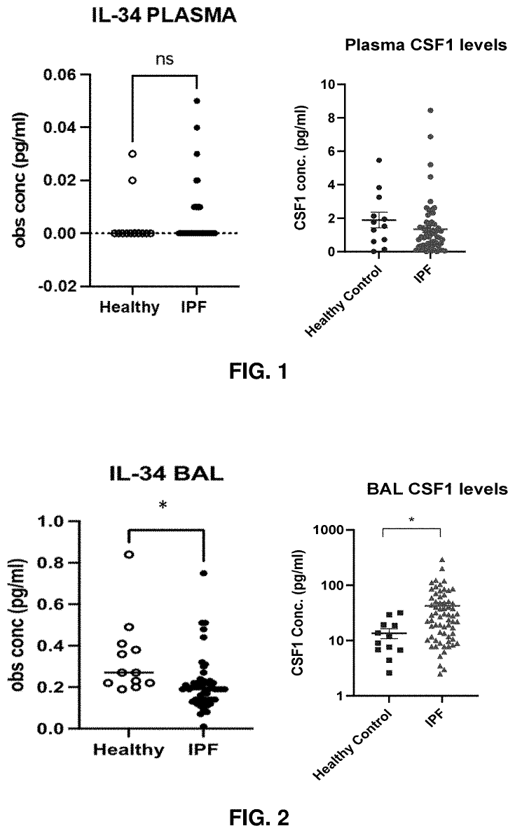

In a first unexpected discovery of the present disclosure, the CSF1 levels in plasma samples of IPF patients were observed to trend lower than in plasma samples from healthy controls (FIG. 1, right panel). Meanwhile, consistent with the earlier reports, the CSF1 levels in BAL fluids were higher in IPF patients (FIG. 2, right panel).

CSF1R has another ligand, IL-34. There is no report, however, concerning IL-34 levels in IPF patients, or generally its use as a biomarker for IPF diagnosis. In the current disclosure, it is discovered that there was apparent difference of plasma IL-34 levels between IPF and healthy controls (FIG. 1, left panel), even though BAL fluid IL-34 levels were lower in IPF patients (FIG. 2, left panel). Nevertheless, in both types of samples, the concentrations of IL-34 were very low, making their detections challenging.

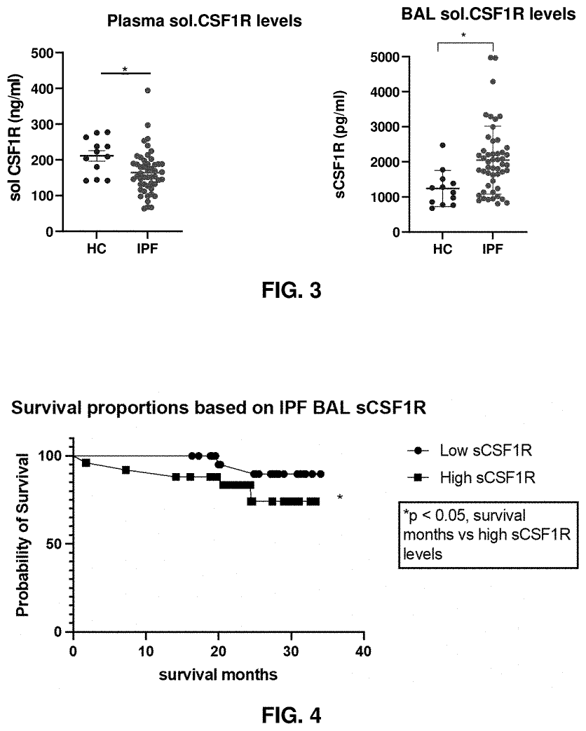

With respect to CSF1R, the literature has discussion of its mRNA levels in various diseases. For the first time, the accompanying example, however, attempted to measure the soluble version of the CSF1R protein (e.g., the extracellular domain without the intracellular portion) in plasma and BAL fluid samples. In another unexpected discovery of the present disclosure, it was found that, relative to healthy controls, average soluble CSF1R levels were decreased in IPF plasma samples (FIG. 3, left panel) and increased in BAL fluid samples (FIG. 3, right panel).

Also interesting, the soluble CSF1R levels in BAL fluids correlated with the disease progression. As shown in FIG. 4, IPF patients having higher BAL soluble CSF1R levels had significantly worse progression free survival (PFS) and were more likely to progress to fatal disease. Yet, even though the plasma soluble CSF1R levels in IPF patients were generally lower than in healthy controls (FIG. 3, left panel), there was a positive correlation between higher plasma soluble CSF1R levels and worse IPF progression within the IPF population.

Importantly, analysis of soluble CSF1R levels in IPF patients (in both plasma and BAL fluids) and the correlation with progression-free survival (PFS) indicated a statistically significant relationship with elevated sol. CSF1R levels in IPF patients and worsening of disease (Table 1). Hence, these results with sol. CSF1R levels reveal that it can be a biomarker for discriminating IPF disease and its severity as well as clinical outcomes.

Therefore, the present data demonstrate that plasma CSF1 and CSF1R levels and BAL fluid CSF1R levels can be suitably used to diagnose IPF, select IPF patients for treatments, and monitor the treatment outcomes.

In one embodiment, therefore, the present disclosure provides a method for identifying a patient as likely having idiopathic pulmonary fibrosis (IPF). In some embodiments, the method entails first measuring the concentration of CSF1 or soluble CSF1R in a blood or a bronchoalveolar lavage (BAL) fluid sample from a patient.

CSF1 and soluble CSF1R proteins in a biological sample, such as plasma, serum or BAL fluid, can be readily measured with methods known in the art. For instance, various known antibodies specific to CSF1 or the CSF1R extracellular domain are available (see, e.g., Tables 1-2) and can be used in an ELISA assay to quantitate their concentrations. Alternatively, mass spectrometry is also well developed for measuring protein concentrations in biological samples.

The concentration of CSF1 or CSF1R can then be used to determine the disease status of the patient. As provided, as compared to healthy individuals, IPF patients have lower CSF1 concentrations and soluble CSF1R concentrations in the plasma, and have higher soluble CSF1R concentrations in the BAL fluids.

Accordingly, in some embodiments, the patient is identified as likely having IPF when the CSF1 concentration in the blood sample is decreased as compared to a reference blood sample from a reference human subject not having IPF. The reference blood sample may be a concurrent sample obtained from a healthy donor, in some embodiments. In a preferred embodiment, the reference blood sample is a historic sample which has a known reference blood CSF1 level. In other words, the known reference blood CSF1 level serves as a reference threshold.

In some embodiments, the threshold concentration is 2 pg/mL. In some embodiments, the threshold concentration is 1.9 pg/mL, 1.8 pg/mL, 1.7 pg/mL, 1.6 pg/mL, 1.5 pg/mL, 1.4 pg/mL, 1.3 pg/mL, 1.2 pg/mL, 1.1 pg/mL, 1.0 pg/mL, 0.9 pg/mL, 0.8 pg/mL, 0.7 pg/mL, 0.6 pg/mL, 0.5 pg/mL, 0.4 pg/mL, 0.3 pg/mL, 0.2 pg/mL, or 0.1 pg/mL, without limitation. Accordingly, in some embodiments, the patient is identified as likely having IPF when the CSF1 concentration in the blood sample is lower than 2 pg/mL, 1.9 pg/mL, 1.8 pg/mL, 1.7 pg/mL, 1.6 pg/mL, 1.5 pg/mL, 1.4 pg/mL, 1.3 pg/mL, 1.2 pg/mL, 1.1 pg/mL, 1.0 pg/mL, 0.9 pg/mL, 0.8 pg/mL, 0.7 pg/mL, 0.6 pg/mL, 0.5 pg/mL, 0.4 pg/mL, 0.3 pg/mL, 0.2 pg/mL, or 0.1 pg/mL.

In some embodiments, the patient is identified as likely having IPF when the soluble CSF1R concentration in the blood sample is decreased as compared to a reference blood sample from a reference human subject not having IPF. The reference blood sample may be a concurrent sample obtained from a healthy donor, in some embodiments. In a preferred embodiment, the reference blood sample is a historic sample which has a known reference blood soluble CSF1R level. In other words, the known reference blood soluble CSF1R level serves as a reference threshold.

In some embodiments, the threshold concentration is 200 ng/mL. In some embodiments, the threshold concentration is 190 ng/mL, 185 ng/mL, 180 ng/mL, 175 ng/mL, 170 ng/mL, 165 ng/mL, 160 ng/mL, 155 ng/mL, 150 ng/mL, 145 ng/mL, 140 ng/mL, 135 ng/mL, 130 ng/mL, 125 ng/mL, 120 ng/mL, 115 ng/mL, 110 ng/mL, 105 ng/mL, 100 ng/mL, 90 ng/mL, 80 ng/mL, 70 ng/mL, 60 ng/mL, or 50 ng/mL, without limitation. Accordingly, in some embodiments, the patient is identified as likely having IPF when the soluble CSF1R concentration in the blood sample is lower than 200 ng/mL, 190 ng/mL, 185 ng/mL, 180 ng/mL, 175 ng/mL, 170 ng/mL, 165 ng/mL, 160 ng/mL, 155 ng/mL, 150 ng/mL, 145 ng/mL, 140 ng/mL, 135 ng/mL, 130 ng/mL, 125 ng/mL, 120 ng/mL, 115 ng/mL, 110 ng/mL, 105 ng/mL, 100 ng/mL, 90 ng/mL, 80 ng/mL, 70 ng/mL, 60 ng/mL, or 50 ng/mL.

In some embodiments, the patient is identified as likely having IPF when the soluble CSF1R concentration in the BAL fluid sample is increased as compared to a reference blood sample from a reference human subject not having IPF. The reference BAL fluid sample may be a concurrent sample obtained from a healthy donor, in some embodiments. In a preferred embodiment, the reference BAL fluid sample is a historic sample which has a known reference BAL fluid soluble CSF1R level. In other words, the known reference BAL fluid soluble CSF1R level serves as a reference threshold.

In some embodiments, the threshold concentration is 1000 pg/mL. In some embodiments, the threshold concentration is 1100 pg/mL, 1200 pg/mL, 1300 pg/mL, 1400 pg/mL, 1500 pg/mL, 1600 pg/mL, 1700 pg/mL, 1800 pg/mL, 1900 pg/mL, 2000 pg/mL, 2100 pg/mL, 2200 pg/mL, 2300 pg/mL, 2400 pg/mL, or 2500 pg/mL, without limitation. Accordingly, in some embodiments, the patient is identified as likely having IPF when the soluble CSF1R concentration in the BAL fluid sample is at least 1000 pg/mL, 1100 pg/mL, 1200 pg/mL, 1300 pg/mL, 1400 pg/mL, 1500 pg/mL, 1600 pg/mL, 1700 pg/mL, 1800 pg/mL, 1900 pg/mL, 2000 pg/mL, 2100 pg/mL, 2200 pg/mL, 2300 pg/mL, 2400 pg/mL, or 2500 pg/mL.

The increased levels of markers can correlate to the disease severity or treatment effects. For instance, soluble CSF1R levels in plasma or BAL samples in IPF patients at or above the average shown here correlated with more severe disease, and worse disease progression.

Accordingly, in one embodiment, a method is provided for monitoring the disease progression in an idiopathic pulmonary fibrosis (IPF) patient. In some embodiments, the method entails measuring the concentration of CSF1 or soluble CSF1R in a blood or a bronchoalveolar lavage (BAL) fluid sample from the IPF patient. The measured concentrations can be then compared to a measurement at an earlier time point for the same patient. In some embodiments, it can be determined that the IPF has worsened when the concentration of soluble CSF1R in the blood sample has increased, or when the concentration of the soluble CSF1R in the BAL fluid sample has increased, as compared to the earlier measurement for the patient.

Another embodiment provides a method for characterizing the severity of idiopathic pulmonary fibrosis (IPF) in an IPF patient, comprising measuring the soluble CSF1R level in a plasma sample from the patient, and characterizing the severity of the IPF including likelihood to progress towards death or >10% FVC loss or lung transplant when the CSF1R level is higher than a reference level. In some embodiments, the reference level is 100 ng/mL (or greater than or equal to 2 after log transformation). In some embodiments, the reference level is 50 ng/mL, 60 ng/mL, 70 ng/mL, 80 ng/mL, 90 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, or 250 ng/mL, without limitation.

Another embodiment provides a method for characterizing the severity of idiopathic pulmonary fibrosis (IPF) in an IPF patient, comprising measuring the soluble CSF1R level in a BAL fluid sample from the patient, and characterizing the severity of the IPF including likelihood to progress towards death or >10% FVC loss or lung transplant when the CSF1R level is higher than a reference level. In some embodiments, the reference level is 2000 pg/mL. In some embodiments, the reference level is 1500 pg/mL, 1600 pg/mL, 1700 pg/mL, 1800 pg/mL, 1900 pg/mL, 2000 pg/mL, 2100 pg/mL, 2200 pg/mL, 2300 pg/mL, 2400 pg/mL, or 2500 pg/mL, without limitation.

### Treatment Methods and Compositions

Compositions and methods of preventing and treating IPF are also provided, which can be employed once a patient is identified as likely to have IPF or to develop IPF.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread of the disease or condition); and/or c) relieving the disease, that is, causing the regression of clinical symptoms.

“Prevention” or “preventing” means any treatment of a disease or condition that causes the clinical symptoms of the disease or condition not to develop. Compositions may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of IPF. The therapeutically effective amount may vary depending on the subject, and disease or condition being treated, the weight and age of the subject, the severity of the disease or condition, and the manner of administering, which can readily be determined by one or ordinary skill in the art.

There are certain known therapies for IPF, including oxygen therapy, pulmonary rehabilitation, and various medications. Example medications include, without limitation, interferon gamma-1β, bosentan, ambrisentan, an anticoagulant, pirfenidone (5-Methyl-1-phenylpyridin-2-one), N-Acetylcysteine (NAC), nintedanib (Methyl (3Z)-3-{[(4-{methyl[(4-methylpiperazin-1-yl)acetyl]amino}phenyl)amino](phenyl)methylidene}-2-oxo-2,3-dihydro-1H-indole-6-carboxylate), a multi-kinase inhibitor (e.g., CSF1 inhibitor), and a CSF1R inhibitor.

CSF1/CSF1R inhibitors are known. Example small molecule CSF1/CSF1R inhibitors include, without limitation, imatinib, nilotinib, and pexidartinib. In some embodiments, the CSF1/CSF1R inhibitor is an antibody.

In some embodiments, the antibody is a human or humanized antibody. An example anti-CSF1R antibody is AM001, which can be prepared as described in PCT application WO 2009/026303 (e.g., antibody 1.2 SM). The epitopes are mainly located at the N-terminus Ig-like loop 1 and Ig-like loop 2 of human CSF1R and requires the presence of both the loop 1 and loop 2 regions. Additional example anti-CSF1 and anti-CSF1R antibodies are provided in Tables A-B.

TABLE A
Example Anti-CSF1R Antibodies

| Name | Other Names | Type |
|---|---|---|
| Emactuzumab | RG7155, or RO5509554 | IgG1 humanized |
| Cabiralizumab | FPA008 | IgG4 humanized |
| Axatilimab | SNDX-6352 | IgG4 humanized |
| IMC-CS4 | LY3022855 | IgG1 human |
| AM001 | 1.2 SM in WO 2009/026303 | IgG2 human |

TABLE B
Example Anti-CSF1 Antibodies

| Name | Other Names | Type |
|---|---|---|
| Lacnotuzumab | MCS110 | IgG1 human |
| PD-0360324 | | IgG2 human |

Emactuzumab (also known as RG7155 and RO5509554) is a clinical stage humanized IgG1 CSF1R targeted antibody designed to target and deplete macrophages in the tumor tissue. It has shown a favorable safety profile in patients and encouraging efficacy for TGCT. Emactuzumab is under investigation in clinical trial NCT01494688—“A Study of RO5509554 as Monotherapy and in Combination with Paclitaxel in Participants With Advanced Solid Tumors.”

Cabiralizumab (also known as FPA008) is under investigation in clinical trial NCT03502330—“APX005M With Nivolumab and Cabiralizumab in Advanced Melanoma, Non-small Cell Lung Cancer or Renal Cell Carcinoma.” Cabiralizumab is a humanized IgG4 anti-CSF1R monoclonal antibody with a single amino acid substitution in the hinge region to prevent hemi-dimer exchange.

IMC-CS4 (also known as LY3022855) is a human IgG1 antibody (mAb) targeting CSF1R. IMC-CS4 is under investigation in clinical trial NCT01346358—“A Study of IMC-CS4 in Subjects With Advanced Solid Tumors.”

Axatilimab (also known as SNDX-6352) is a humanized, full-length IgG4 antibody with high affinity to CSF-1R. Axatilimab affects the migration, proliferation, differentiation, and survival of monocytes and macrophages by binding to CSF-1R and blocking its activation by its two known ligands, CSF-1 and IL-34. Axatilimab is currently being evaluated in a Phase 1/2 clinical trial in patients with cGVHD.

Lacnotuzumab (also known as MCS110) is a high-affinity human engineered IgG1 anti-CSF1 antibody that blocks the ability of CSF1R to drive proliferation in responsive cells. Lacnotuzumab is under investigation in clinical trial NCT01643850—“MCS110 in Patients With Pigmented Villonodular Synovitis (PVNS).”

PD-0360324 is a fully human immunoglobulin G2 monoclonal antibody against CSF1 investigated for treating cutaneous lupus erythematosus (CLE). It is also being tested for its combination with Cyclophosphamide in treating patients with recurrent high-grade epithelial ovarian, primary peritoneal, or fallopian tube cancer.

Accordingly, in some embodiments, a method is provided for treating an idiopathic pulmonary fibrosis (IPF). In some embodiments, the method entails administering a CSF1 inhibitor or a CSF1R inhibitor to a patient that (a) has a blood CSF1 concentration lower than a reference blood CSF1 concentration from a reference human subject not having IPF, (b) has a blood soluble CSF1R concentration lower than a reference blood soluble CSF1R concentration from a reference human subject not having IPF, or (c) has a BAL fluid soluble CSF1R concentration higher than a reference BAL fluid soluble CSF1R concentration from a reference human subject not having IPF.

In some embodiments, the patient has a CSF1 concentration in the blood sample that is lower than 2 pg/mL, 1.9 pg/mL, 1.8 pg/mL, 1.7 pg/mL, 1.6 pg/mL, 1.5 pg/mL, 1.4 pg/mL, 1.3 pg/mL, 1.2 pg/mL, 1.1 pg/mL, 1.0 pg/mL, 0.9 pg/mL, 0.8 pg/mL, 0.7 pg/mL, 0.6 pg/mL, 0.5 pg/mL, 0.4 pg/mL, 0.3 pg/mL, 0.2 pg/mL, or 0.1 pg/mL.

In some embodiments, the patient has a soluble CSF1R concentration in the blood sample that is lower than 200 ng/mL, 190 ng/mL, 185 ng/mL, 180 ng/mL, 175 ng/mL, 170 ng/mL, 165 ng/mL, 160 ng/mL, 155 ng/mL, 150 ng/mL, 145 ng/mL, 140 ng/mL, 135 ng/mL, 130 ng/mL, 125 ng/mL, 120 ng/mL, 115 ng/mL, 110 ng/mL, 105 ng/mL, 100 ng/mL, 90 ng/mL, 80 ng/mL, 70 ng/mL, 60 ng/mL, or 50 ng/mL.

In some embodiments, the patient is has a soluble CSF1R concentration in the BAL fluid sample that is at least 1000 pg/mL, 1100 pg/mL, 1200 pg/mL, 1300 pg/mL, 1400 pg/mL, 1500 pg/mL, 1600 pg/mL, 1700 pg/mL, 1800 pg/mL, 1900 pg/mL, 2000 pg/mL, 2100 pg/mL, 2200 pg/mL, 2300 pg/mL, 2400 pg/mL, or 2500 pg/mL.

Treatment monitoring methods are also provided. In one embodiments, a method is provided for monitoring the effect of a treatment of an idiopathic pulmonary fibrosis (IPF) patient, which entails measuring the concentration of CSF1 or soluble CSF1R in a blood or a bronchoalveolar lavage (BAL) fluid sample from the IPF patient, and determining that the treatment is effective when the concentration of CSF1 or soluble CSF1R in the blood sample has increased, or when the concentration of the soluble CSF1R in the BAL fluid sample has decreased, as compared to an earlier measurement for the patient during or before the treatment.

In some embodiments, the method determines that the treatment is not effective when the concentration of CSF1 or soluble CSF1R in the blood sample has not increased, or when the concentration of the soluble CSF1R in the BAL fluid sample has not decreased, as compared to an earlier measurement for the patient during or before the treatment.

### Kits and Packages

The methods described herein may be performed, for example, by utilizing pre-packaged diagnostic kits, such as those described below, comprising at least one probe or primer nucleic acid described herein, which may be conveniently used, e.g., to determine whether a subject has or is at risk of developing a disease such as IPF.

Diagnostic procedures can be performed with blood (e.g., plasma and serum) samples and/or bronchoalveolar lavage (BAL) fluid samples obtained from a patient. The detection can be made with antibodies specific to CSF1 or CSF1R.

In one embodiment, provided is a kit or package useful for diagnosing IPF, comprising antibodies for measuring the level of CSF1 and/or CSF1R. In some embodiments, the kit or packages includes antibodies to both CSF1 and CSF1R, and reagents for ELISA assays.

In one embodiment, a kit further includes instructions for use. In one aspect, a kit includes a manual comprising reference gene expression levels.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Biomarkers for Idiopathic Pulmonary Fibrosis

This example tested the levels of various proteins in different biological samples obtained from patients of idiopathic pulmonary fibrosis (IPF) and those from healthy individuals as control.

The biological samples included plasma and bronchoalveolar lavage (BAL) fluid. The samples were retrieved from the IPF patients and healthy individuals at the Royal Brompton Hospital (RBH).

Bio-Techne Luminex (Thermo Fisher Scientific) was used to measure the levels of IL-34 and CSF1 per manufacturer's protocol. The results were further confirmed with MSD U-PLEX Human M-CSF (Meso Scale Diagnostics, LLC) per manufacturer's protocol. The levels of CSF1R/CD115 were measured with RayBiotech Human M-CSF1R ELISA (ELH-MCSFR-1) per manufacturer instructions.

### Results

CSF1R has two ligands, IL-34 and CSF1. Their plasma levels were measured in the samples. As shown in FIG. 1, the levels of both IL-34 and CSF1 were not significantly different between healthy and IPF subjects. Interestingly, the CSF1 levels were actually lower in IPF vs healthy subjects. This is surprising, as Fraser et al. (Front Immunol, 2021 Mar. 5; 12:623430) has recently reported elevated serum CSF1 levels in IPF patients (N=37, ˜1000 pg/ml) vs controls (N=28, ˜250 pg/ml).

Interestingly, in BAL fluid samples, the IL-34 levels were reduced in IPF subjects as compared to heavy ones, while the levels of CSF1 were significantly increased in IPF subjects (FIG. 2).

The concentrations of the soluble CSF1R (extracellular domain) were also measured in the plasma and BAL fluid samples. In plasma samples, the soluble CSF1R (sol. CSF1R, or sCSF1R) levels were lower in IPF patients relative to healthy controls, but in the BAL fluid sample, the soluble CSF1R levels were significantly higher in IPF patients, as compared to healthy controls (FIG. 3).

The levels of soluble CSF1R in BAL fluid samples were then analyzed against the disease progression records of the patients. As shown in FIG. 4, IPF patients having higher BAL soluble CSF1R levels had significantly worse (odds ratio (OA) 3.2, p=0.025) 2-year progression free survival (PFS) and 1-year PFS as shown in Table 1. Also, IPF patients with higher BAL sCSF1R levels were more likely to progress to fatal disease (p<0.05). Similar correlations were observed between plasma soluble CSF1R levels and IPF progression, even though the plasma soluble CSF1R levels in IPF patients were generally lower than in healthy controls.

TABLE 1
Correction between plasma soluble CSF1R levels
and 1-year progression free survival (PFS)

| Protein | Odds Ratio (95% CI) | P-value |
|---|---|---|
| sol. CSF1R | 2.9 | 0.045 |

PFS = death or >10% decline in lung forced vital capacity (FVC) or lung transplant

Further, as shown in Table 2, the log₁₀ transformation sol. CSF1R levels (in ng/ml) in IPF patients can be utilized to ascertain a subpopulation difference in disease progression and that those >1=2.0 have nearly 3-fold greater progression than those <2.0.

TABLE 2
Prevalence of one-year progression between pg/ml
log transformed plasma sCSFR1 threshold

| | Progression |
|---|---|
| sCSFR1 Level | % |
| <2.0 | 20 |
| >=2.0 | 57 |

Progression = death or >10% decline in lung forced vital capacity (FVC) or lung transplant

To the best knowledge of the inventors, soluble CSF1R levels have not been examined before for IPF patients, much less to be correlated to disease progression.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for identifying a patient as likely having idiopathic pulmonary fibrosis (IPF), comprising:
measuring the concentration of CSF1 or soluble CSF1R in a blood or a bronchoalveolar lavage (BAL) fluid sample from a patient; and
identifying the patient as likely having IPF when the CSF1 concentration or soluble CSF1R concentration in the blood sample is decreased as compared to a reference blood sample from a reference human subject not having IPF, or when the soluble CSF1R concentration in the BAL fluid sample is increased as compared to a reference BAL fluid sample from a reference human subject not having IPF.

2. The method of claim 1, further comprising treating the patient identified as likely having IPF.

3. The method of claim 2, wherein the patient is treated with an oxygen therapy, pulmonary rehabilitation, or an agent selected from the group consisting of interferon gamma-1β, bosentan, ambrisentan, an anticoagulant, pirfenidone, N-Acetylcysteine (NAC), nintedanib, a multi-kinase inhibitor, and a CSF1R inhibitor.

4. The method of claim 3, wherein the patient is treated with an anti-CSF1 antibody or an anti-CSF1R antibody.

5. The method of claim 1, wherein the patient is identified as likely having IPF when the soluble CSF1R concentration in the BAL fluid sample is increased as compared to the reference BAL fluid sample.

6. The method of claim 5, wherein the patient is identified as likely having IPF when the soluble CSF1R concentration in the BAL fluid sample is greater than 1500 pg/mL.

7. The method of claim 5, wherein the patient is identified as likely having IPF when the soluble CSF1R concentration in the BAL fluid sample is greater than 1800 pg/mL, preferably greater than 2000 pg/mL.

8. The method of claim 1, wherein the blood sample is a plasma or serum sample.

9. The method of claim 8, wherein the patient is identified as likely having IPF when the soluble CSF1R concentration in the blood sample is decreased as compared to the reference blood sample from the reference human subject not having IPF.

10. The method of claim 9, wherein the patient is identified as likely having IPF when the soluble CSF1R concentration in the blood sample is lower than 200 ng/mL.

11. The method of claim 9, wherein the patient is identified as likely having IPF when the soluble CSF1R concentration in the blood sample is lower than 180 ng/mL, preferably lower than 160 pg/mL.

12. The method of claim 8, wherein the patient is identified as likely having IPF when the blood CSF1 concentration is decreased as compared to the reference blood sample from the reference human subject not having IPF.

13. The method of claim 12, wherein the patient is identified as likely having IPF when the CSF1 concentration in the blood sample is lower than 1.8 pg/mL.

14. The method of claim 12, wherein the patient is identified as likely having IPF when the CSF1 concentration in the blood sample is lower than 1.6 pg/mL, preferably lower than 1.5 pg/mL.

15. A method for treating an idiopathic pulmonary fibrosis (IPF), comprising administering a CSF1 inhibitor or a CSF1R inhibitor to a patient that
(a) has a blood CSF1 concentration lower than a reference blood CSF1 concentration from a reference human subject not having IPF,
(b) has a blood soluble CSF1R concentration lower than a reference blood soluble CSF1R concentration from a reference human subject not having IPF, or
(c) has a BAL fluid soluble CSF1R concentration higher than a reference BAL fluid soluble CSF1R concentration from a reference human subject not having IPF.

16. The method of claim 15, wherein the CSF1 inhibitor is an anti-CSF1 antibody, or the CSF1R inhibitor is an anti-CSF1R antibody.

17. A method for monitoring the disease progression in an idiopathic pulmonary fibrosis (IPF) patient, comprising measuring the concentration soluble CSF1R in a blood or a bronchoalveolar lavage (BAL) fluid sample from the IPF patient, and determining that the IPF has worsened when the concentration of soluble CSF1R in the blood or BAL fluid sample has increased.

18. The method of claim 17, wherein the treatment is with a CSF1 inhibitor or a CSF1R inhibitor.
